(19) United States
(12) Patent Application Publication
(10) Pub. No.: US 2010/0179219 A1
(43) Pub. Date: Jul. 15, 2010

(54) Plane Tree Cream, Method for the Production Thereof and Use

(75) Inventor: Ulrich Meyer, Berlin (DE)

Correspondence Address: HAYES SOLOWAY P.C., 3450 E. SUNRISE DRIVE, SUITE 140, TUCSON, AZ, 85718, US

(73) Assignee: WALA-HEILMITTEL GMBH, Boll (DE)

(21) Appl. No.: 12/377,206

(22) PCT Filed: Aug. 13, 2007
(86) PCT No.: PCT/EP07/07140

§ 371 Date: Feb. 11, 2009

(30) Foreign Application Priority Data
Aug. 17, 2006 (DE) 102006038471.7-41

(51) Int. Cl. A61K 31/19; A61P 17/06; A61P 17/10; A61P 17/12

(52) U.S. Cl. 514/557

(57) ABSTRACT

In order to prevent betulinic acid from crystallizing in a galenic preparation containing water, a plane tree cream in the form of a stable emulsion containing oil and water is proposed, which is characterized by a content of 2 to 10% by weight of a plane tree extract obtained from the bark of plane trees, 0.1 to 9.9% by weight of glycerol, 0.1 to 1.9% by weight of a gelatinizing agent and 5 to 15% by weight of a preservative. The plane tree extract used is obtained by extracting plane tree bark with a low alkanol. The plane tree cream is used for the topical treatment of the skin, including the mucous membranes.

The invention relates to a plane tree cream in the form of a stable emulsion containing oil and water, and to a method for the production thereof, in which first an extract is produced from plane tree bark and then mixed with oil, water and further additives to form an emulsion. The plane tree cream is used for the topical treatment of the skin, including the mucous membranes.

About ten species of the plane tree (Platanus), the sole genus of the plane tree family (Platanaceae), can be found worldwide. The characteristic feature is its pale plate-like bark which peels off by itself over large areas and which has always been of interest to pharmacologists. In Central Europe, the maple-leaved plane tree (Platanus acerifolia) is found most frequently, a hybrid of the American plane tree (Platanus occidentalis) and the plane tree from Asia Minor (Platanus orientalis).

As in the case of birch bark, betulinic acid and other triterpene derivatives are also obtained from plane tree bark.

Document DE 197 13 768 B4 discloses for example the production of betulinic acid from the dry pulverized bark of the Platanus acerifolia by extraction with dichloromethane, chloroform and diethyl ether, the plane tree extract being re-crystallized from methanol after having drawn off the specified solvents.

It is known from document EP 1 267 900 B1 to extract triterpenes from vegetable material, preferably from birch bark, with a supercritical carbon dioxide or with low-boiling hydrocarbons such as pentane, hexane or heptane at a pressure of 1 to 300 bar and a temperature of 50 to 200° C. after having extracted or washed the vegetable material with a nonsolvent for the triterpenes to first eliminate undesired byproducts and increase the purity of the extract.

Document EP 1 267 900 B1 further discloses an emulsion which contains the triterpene-containing vegetable extract thus obtained, oil, water and, if necessary, further additives, the triterpenes contained being not only pharmacologically active but being also intended to act themselves as emulsifiers for the formation and stabilization of the emulsion and at the same time as the only preservative.

Betulinic acid, also referred to as 3β-hydroxy-20(29)-lupene-28-acid, and platanic acid have the following general formula

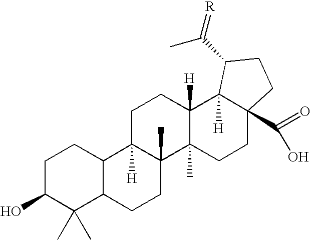

wherein R is a methylene group (CH₂) in the case of the betulinic acid and an oxygen atom in the case of the platanic acid. Pyracrenic acid is the 3-((3-(3,4-dihydroxyphenyl)-1-oxo-2-propenyl)oxy) derivative of betulinic acid.

Betulinic acid has a wide pharmacological spectrum having, among other things, a significant effect against HIV viruses and human melanoma cells as a result of phosphokinase C inhibition [J. Med. Chem. 39, 1016, 1056, 1069 (1996)].

Up to now, the fact that the acids crystallize upon contact with minimum quantities of water and therefore cannot be incorporated into a stable emulsion containing an oily and an aqueous phase and having a constant active agent content that can be standardized has constituted an obstacle to a topical application of a plane tree extract containing betulinic acid and platanic acid.

It is therefore the object of the invention to provide an aqueous galenic preparation for a plane tree extract containing platanic acid and betulinic acid, the preparation preventing the crystallization of the active triterpene constituents of the extract in spite of its water content, having hydrophilic properties and being suitable for the topical treatment of the skin and the mucous membranes.

According to the invention, this object is achieved by the plane tree cream having the features of claim 1 and by a method in which an extract is produced from plane tree bark and is mixed with oil, water, glycerol, a gelatinizing agent and a preservative to form an emulsion, the plane tree bark being extracted with an alcohol which is selected from the group consisting of the alkanols having 1 to 5 carbon atoms and mixtures thereof.

It surprisingly appeared that using alkanols of the group methanol, ethanol, propanols, butanols, pentanols and the mixtures of these alcohols results in an extract which together with oil, water, glycerol, a gelatinizing agent and a preservative forms a plane tree cream in the form of a stable emulsion in which betulinic acid and platanic acid do not crystallize despite the water content of the emulsion. It is supposed that the special alcohols used for the extraction produce such a complex mixture of ingredients of the plane tree bark that this mixture together with the additives glycerol, the gelatinizing agent and the preservative stops the crystallizability of the specified acids in the presence of water.

The plane tree cream according to the invention contains 2 to 10% by weight of the plane tree extract, 0.1 to 9.9% by weight of glycerol, 0.1 to 1.9% by weight of a gelatinizing agent and 5 to 15% by weight of a preservative, preferably in the form of an alkanol having 2 to 5 C atoms, more preferably in the form of ethanol.

Germ contamination tests have shown that the addition of a preferably alcoholic preservative is indispensable to prevent the growth of germs in the cream after a contamination by ubiquitously appearing germs such as, e.g., Escherichia coli, Pseudomonas aeruginosa, Staphylococcus aureus, Candida albicans and Aspergillus niger. Details to the contrary, as are found in document EP 1 267 900 B1, could not be verified.

In addition to platanic acid and betulinic acid, the plane tree extract in the plane tree cream according to the invention preferably also contains 29-hydroxyplatanic acid, betulonic acid and pyracrenic acid.

The plane tree cream according to the invention can be present in the form of an oil-in-water emulsion (O/W emulsion) or in the form of a water-in-oil emulsion (W/O emulsion). In both cases, the outer phase constitutes at most 70% of the emulsion, and the inner phase at least 20%.

Preferably, the plane tree cream contains as gelatinizing agent a cellulose derivative or starch derivative, poly(meth)acrylate, xanthan gum, alginate, carrageenan, galactoarabinan or a mixture of these substances or substance classes.

A particular advantage of the plane tree cream according to the invention is that it can be free of added foreign emulsifiers because in dermatological terms, the conventional emulsifiers present a problem as potential allergens. It is supposed that the constituents of the plane tree extract act themselves as emulsifiers; they might possibly not have this effect as individual substances, but at least in their special combination produced by the extracting agents used.

The plane tree extract contained in the plane tree cream preferably contains at least 15% platanic acid and at least 10% betulinic acid, and is particularly preferably a dry extract.

In a particularly preferred embodiment of the invention, the three constituents platanic acid, 29-hydroxyplatanic acid and betulinic acid together constitute at least 35% by weight of the dry extract.

The oil phase of the emulsion is preferably formed by a vegetable oil or by a mixture of vegetable oils, with avocado oil, almond oil or mixtures thereof being particularly preferably used.

When producing the plane tree cream according to the invention, dried and pulverized plane tree bark is preferably used as a starting material for the plane tree extract and is particularly preferably extracted with one of the specified alcoholic solvents using a flow extractor or a Soxhlet extractor. Since the solvent is heated to boiling under reflux in the Soxhlet extractor and the condensate dripping from the cooler flows back through the material to be extracted, the extraction is here carried out at the boiling temperature of the alcohol used or slightly below this boiling temperature. In the flow extractor, the extraction is performed at room temperature.

Preferably, the extract is dried by drawing off the alcoholic extracting agent, for example at the rotary evaporator, and the dry extract thus obtained is incorporated into the emulsion of the remaining constituents.

Preferably, the emulsion is formed without the addition of an emulsifier, the term “emulsifier” referring here to a separate additive and not to the natural constituents of the plane tree extract which could themselves have emulsifying properties.

As already mentioned, a cellulose derivative or starch derivative, poly(meth)acrylate, xanthan gum, alginate, carrageenan, galactoarabinan or a mixture of these materials or material classes is preferably used as gelatinizing agent. Here, the gelatinizing agent is used in an amount of less than 2% by weight of the total mass.

Advantageously, an alkanol having 2 to 5 C atoms is used as preservative, particularly preferably ethanol, more specifically preferably in an amount of 5 to 15% by weight of the total mass.

According to the invention, part of the alcoholic plane tree bark extract can also be replaced with a carbon dioxide extract of the plane tree bark, i.e. with an extract obtained with supercritical CO₂ as extracting agent. The carbon dioxide extract, the production of which with an alcoholic entraining agent is also permissible, can constitute 5 to 50% of the total solids of the plane tree bark extract. This embodiment is of particular economic importance since it is known that pure carbon dioxide extracts of the plane tree bark cannot take the form of a stable emulsion. However, surprisingly, these carbon dioxide extracts which are otherwise unusable for the production of a plane tree cream can be co-emulsified in a stable form if they are used together with the special alcoholic plane tree bark extract employed here.

The plane tree cream according to the invention is particularly suitable for the topical treatment of the skin, including the mucous membranes, more specifically preferably in case of actinic keratoses, psoriasis, warts and tumors of humans and mammals. Due to the stability of the emulsion, a good standardization of the plane tree cream to a desired triterpene content is possible.

Whereas pure betulinic acid cannot take the galenic form of a stable emulsion containing water due to its susceptibility to crystallization even in the presence of small quantities of water, this is surprisingly achieved with the invention without difficulty and in a very cost-effective manner. Without intending to be bound to a theory, it is supposed that this primarily results from the fact that in the plane tree extract used in accordance with the invention, the betulinic acid is not present in a pure form but in a very special mixture with other ingredients of the plane tree bark, the special composition of the mixture being a result of the solvents used for the extraction.

1. A plane tree cream in the form of a stable emulsion containing oil and water, characterized by a content of
2 to 10% by weight of a plane tree extract obtained from the bark of plane trees,
0.1 to 9.9% by weight of glycerol,
0.1 to 1.9% by weight of a gelatinizing agent, and
5 to 15% by weight of a preservative.

2. The plane tree cream according to claim 1, characterized in that the plane tree extract contains platanic acid, 29-hydroxyplatanic acid, betulinic acid, betulonic acid and pyracrenic acid.

3. The plane tree cream according to claim 2, characterized in that the plane tree extract contains at least 15% by weight platanic acid and at least 10% by weight betulinic acid.

4. The plane tree cream according to claim 3, characterized in that the plane tree extract is a dry extract.

5. The plane tree cream according to claim 4, characterized in that the constituents platanic acid, 29-hydroxyplatanic acid and betulinic acid together constitute at least 35% by weight of the dry extract.

6. The plane tree cream according to claim 1, characterized in that the emulsion is an O/W emulsion the aqueous phase of which constitutes at most 70% and the oil phase of which constitutes at least 20%.

7. The plane tree cream according to claim 1, characterized in that the emulsion is a W/O emulsion the oil phase of which constitutes at most 70% and the aqueous phase of which constitutes at least 20%.

8. The plane tree cream according to claim 1, characterized in that the gelatinizing agent is a cellulose derivative or starch derivative, poly(meth)acrylate, xanthan gum, alginate, carrageenan, galactoarabinan or a mixture thereof.

9. The plane tree cream according to claim 1, characterized in that the preservative is an alkanol having 2 to 5 C atoms.

10. The plane tree cream according to claim 9, characterized in that the preservative is ethanol.

11. The plane tree cream according to claim 1, characterized in that it is free of separately added emulsifiers.

12. The plane tree cream according to claim 1, characterized in that it is free of crystalline betulinic acid.

13. The plane tree cream according to claim 1, characterized in that the oil is a vegetable oil or a mixture of vegetable oils.

14. The plane tree cream according to claim 13, characterized in that the oil is avocado oil, almond oil or a mixture thereof.

15. A method of producing the plane tree cream according to claim 1, in which an extract is produced from plane tree bark and mixed with oil, water, glycerol, a gelatinizing agent and a preservative to form an emulsion, the plane tree bark being extracted with an alcohol which is selected from the group consisting of methanol, ethanol, propanols, butanols, pentanols and mixtures thereof.

16. The method according to claim 15, characterized in that the plane tree bark is dried and pulverized before extraction.

17. The method according to claim 15, characterized in that the extraction is carried out using a flow extractor or a Soxhlet extractor.

18. The method according to claim 15, characterized in that the extract is dried by drawing off the alcoholic extracting agent and in that the dried extract obtained thereafter is incorporated into the emulsion of the remaining constituents.

19. The method according to claim 15, characterized in that the emulsion is formed without the addition of an emulsifier.

20. The method according to claim 15, characterized in that a cellulose derivative or starch derivative, poly(meth)acrylate, xanthan gum, alginate, carrageenan, galactoarabinan or a mixture thereof is used as gelatinizing agent.

21. The method according to claim 20, characterized in that the gelatinizing agent is used in an amount of less than 2% by weight of the total mass.

22. The method according to claim 15, characterized in that an alkanol having 2 to 5 C atoms is used as preservative.

23. The method according to claim 22, characterized in that ethanol is used as preservative.

24. The method according to claim 22, characterized in that the preservative is used in an amount of 5 to 15% by weight of the total mass.

25. The method according to claim 15, characterized in that the alcoholic plane tree bark extract is mixed with a portion of plane tree bark extract which was obtained using supercritical carbon dioxide as extracting agent.

26. The method according to claim 25, characterized in that the carbon dioxide extract constitutes 5 to 50% of the total solids of the plane tree bark extract.

27. A method for treating the skin and/or the mucous membrane in case of actinic keratoses, psoriasis, warts and tumors, which comprises applying to the skin or mucous membrane to be treated, plane tree cream of claim 1.
